(19) United States
(12) Patent Application Publication
(10) Pub. No.: US 2012/0101342 A1
(43) Pub. Date: Apr. 26, 2012

(54) PEDIATRIC TISSUE ILLUMINATOR

(76) Inventors: Thomas P. Duffy, Maricopa, AZ (US); Gerald T. Harder, Chandler, AZ (US); Kevin Mori, Chandler, AZ (US)

(21) Appl. No.: 13/276,321

(22) Filed: Oct. 19, 2011

Related U.S. Application Data
(60) Provisional application No. 61/405,532, filed on Oct. 21, 2010.
(60) Provisional application No. 61/405,543, filed on Oct. 21, 2010.

(51) Int. Cl. A61B 1/06

(52) U.S. Cl. 600/249

(57) ABSTRACT

A pediatric tissue illuminator includes a semi-rigid lighting head removably connected to a powered base unit for illuminating an infant's tissues while stabilizing a limb for venipuncture. The lighting head includes a series of LEDs and is placed behind an infant's arm, thereby directing light therethrough for enhanced viewing. A printed circuit board in the lighting head routes electrical conductors to the LEDs. A translucent soft covering is disposed between the printed circuit board and the infant's body. The base unit includes a control circuit for varying the application of electrical power to the lighting head via an electrical cable. The arm board/light head is inexpensive enough to be disposable. Alternatively, a re-useable light head may be used with disposable semi-rigid housings.

### CROSS REFERENCE TO RELATED APPLICATIONS

This application claims the benefit of the earlier filing date of U.S. provisional patent application No. 61/405,532, entitled “Pediatric Tissue Illuminator”, filed on Oct. 21, 2010, by the same inventors named herein, pursuant to 35 USC §119(e). This application also claims the benefit of the earlier filing date of U.S. provisional patent application No. 61/405,543, entitled “Medical Imaging Device”, filed on Oct. 21, 2010, by the same inventors named herein, pursuant to 35 USC §119(e).

### FIELD OF THE INVENTION

The present invention relates to the field of medical imaging. More particularly, the present invention relates to tissue illumination and pediatric medicine.

### BACKGROUND OF THE INVENTION

In order to safely and effectively administer intravenous (IV) lines or draw blood from a patient, it is critical for the health care provider to be able to locate suitable veins. In many instances, experienced medical personnel are able to locate veins by feel or by visual inspection under normal lighting conditions. However, in some cases, the use of sophisticated medical devices is necessary in order to assist in the location of veins on a patient.

Imaging of subcutaneous veins by using LED light to illuminate tissue is generally known in the medical industry. LED based trans-illumination devices use light from the visible spectrum, and shine it into the tissue that is to be investigated. These devices have many important benefits including simplicity, low cost, and portability. However, such devices are not always effective for locating veins due to the high variability in the absorption and scattering of visible light within human tissue of patients. Difficulties often result from variations in skin tones, body fat, and other physical characteristics.

Typically, LED-based trans-illumination devices use colors in the near-infrared portion of the visible spectrum, such as red or orange, in order to provide the best view of subcutaneous veins. Some trans-illumination devices also use white light in conjunction with red or orange in an attempt to augment the effectiveness of the longer wavelength red or orange light alone. However, LED trans-illuminators are not always effective for locating subcutaneous veins on a given patient for a variety of reasons.

For example, veins in patients with darker skin color are known to be more difficult to locate using trans-illumination due to the absorption of light by the melanin which creates darker skin tones. Additionally, veins in obese patients are also known to be more difficult to trans-illuminate, possibly due to the increased amount of fatty tissue surrounding the vein, which serves to scatter or absorb the light and obscure the subcutaneous veins. It should be noted as well that extensive clinical studies have not been completed to quantitatively assess the effectiveness of various light wavelengths for the purposes of vein location. Ideally then, effective trans-illumination on a large patient population may be best accomplished by attempting to try a variety of colors at varying intensities to see which works best for a given case.

LED based trans-illumination devices that are currently available are typically battery-powered, often using either dry cell batteries (the common AA or AAA batteries used in consumer electronic devices) or rechargeable batteries such as lithium ion (LiOn). Such devices may be provided in handheld form so as to be easily used and carried in a lab coat pocket. A single piece construction is most common, with the LEDs housed in the same plastic enclosure as the batteries and electronics used to drive the LEDs. The advantage of the single piece construction is that it is compact; the disadvantage is that the entire unit must be held or otherwise secured against the patient's skin while the vein is located and the IV insertion or other procedure takes place.

Trans-illumination devices using a base unit and a remote LED lighting unit are also available. The base unit may be either battery powered or powered from an AC wall source. The lighting unit is either LED or halogen-based, depending on the model, and is connected to the base by a cord. In either case, however, a single, dedicated lighting unit is provided with the base unit.

The primary reason for a health care provider to locate veins on a patient is to perform a venipuncture to insert an intravenous line or to collect a blood sample. Clearly then, electronic devices used to assist in vein location may be exposed to blood or other bodily fluids. It is therefore highly desirable for the vein location device to be either sterilizable using common techniques used in hospitals and medical offices, or to be designed for one time use. Current vein location devices are relatively expensive, and are too costly to be considered disposable, i.e., they are intended for multiple uses.

An autoclave is often used to sterilize medical equipment in hospitals and medical offices. An autoclave uses a combination of temperature, pressure, and humidity to create a sterile environment. Typically, an autoclave may expose its contents to heat at 125 degrees Centigrade for about 45 minutes at a high relative humidity. Generally, currently-available battery-operated LED trans-illumination devices are not designed to withstand such conditions, and heating such equipment within an autoclave either renders such devices non-functional or significantly reduces their useful life.

Infants present a special case of tissue illumination. Because the infant's limbs are so small, tissues may be illuminated by shining light directly through them, rather than by illuminating them with light from the upper surface. For example, when illuminating the arm of an infant during placement of an intravenous (IV) line, light may be directed into the infant's arm from below, i.e., beneath the infant's arm. Sufficient illumination can pass through the infant's limb without being absorbed, such that the subcutaneous features are easily visible.

Others have attempted to adapt vein illuminators for pediatric applications. For instance, in U.S. Patent Application Publ. No. 2008/0015663, Mullani describes the use of an adapter to modify a prior art LED illumination device for pediatric use. U.S. Pat. No. 7,431,695, entitled “Neonatal Transilluminator Apparatus”, to Creaghan discloses an illumination device adapted to the skin texture of small children, and provides an illumination system capable of shining through an infant's limb. This patent appears to describe a device commercially available from Venoscope, LLC of LaFayette, La. under the product name “Neonatal Transilluminator”. However, Creaghan's illumination device is small and difficult to use with an uncooperative child.

Since the simple LED-based trans-illumination devices described above are not always effective, alternate technologies are also employed for the purpose of vein illumination. Imaging devices using infrared (IR) radiation are available, but such systems are inherently more costly and difficult to use. Since infrared wavelengths are not visible to the human eye, electronic detectors must be employed, with the resulting captured image projected onto a display or back onto the patient's skin. Thus, IR systems require much more sophisticated electronics, and furthermore require the health care provider to view a projected image of the tissue, rather than the tissue itself.

Alternatively, ultrasound imaging technology may also be used to locate veins. However, like the IR systems discussed above, ultrasound technology is far more costly and difficult to use than a simple LED-based trans-illumination device. Furthermore, while ultrasound technology is able to image deeply within human tissue, it is often ineffective for imaging near the skin's surface. Thus, ultrasound systems may in fact have difficulty imaging the most accessible veins near the skin's surface.

Pediatric splints, or arm boards, are known to those skilled in the art to stabilize, and prevent bending, of the limb of an infant or small child. Such pediatric arm boards are available, for example, under the trademark Pedi-Boards® from Pedicraft, Inc. of Jacksonville, Fla. Since infants will instinctively resist any medical procedure, it is often desirable to secure the infant's limb in some fashion before attempting a medical procedure, such as insertion of an intravenous line. In practice, health care providers will often use a small arm board when placing an intravenous line in an infant. See, for example, Pinsky, and Young, “Performing pediatric venipuncture without fear”, Nursing, October 1997. The arm board is placed beneath the infant's arm. Then, the venipuncture is performed, and the intravenous line is set in place. Lastly, the IV line is secured with tape which is placed over the IV line and around the arm board, in order to minimize discomfort to the infant. However, pediatric splints do not include a source of light to illuminate the infant's limb. In fact, such pediatric splints are typically opaque, and the presence of a pediatric splint actually precludes the use of a source of back-light for illuminating the infant's limb.

It is therefore an object of the present invention to provide a pediatric tissue illumination device useful for small children and infants.

It is a further object of the present invention to provide such an illumination device that can also function as a restraint or splint.

It is a yet further object of the present invention to provide such an illumination device that is portable and easy-to-use.

Still another object of the present invention is to provide such an illumination device that can be used to locate veins or other tissues on a larger percentage of patients than currently-known devices.

Yet another object of the present invention is to provide such an illumination device wherein the cost of the device is low enough to facilitate single-use applications, effectively making the illumination head of the device disposable.

Another object of the present invention is to provide such an illumination device wherein the device may be powered by a simple, low cost driving apparatus.

These and other objects of the present invention will become more apparent to those skilled in the art as the description thereof proceeds.

### SUMMARY OF THE INVENTION

A tissue illumination device is described which is particularly well-adapted for pediatric use, and which facilitates viewing of tissues that lie below the skin of a portion of an infant's body. In a preferred embodiment of the invention, a battery-powered base unit is releasably coupled to a lighting head by an electrical cable, e.g., using standard electrical connectors. The battery housed within the base unit is preferably re-chargeable, and provides a source of electrical power for the lighting head. The lighting head includes a semi-rigid elongated board supporting a plurality of electrical conductors for conducting electrical signals. The lighting head may be easily connected to, and disconnected from, the base unit, allowing a nurse or medical technician to quickly and easily switch from one lighting head to another.

In the preferred embodiment, the lighting head is provided in the form of a pediatric splint, or arm board, for pediatric tissue illumination while simultaneously stabilizing the limb against movement or bending. Each lighting head is preferably disposable for one time use. In its preferred form, the lighting head includes and supports a series of light-emitting devices that are preferably light-emitting diodes, or “LEDs”. These LEDs preferably emit light in a low energy portion of the visible light spectrum, i.e., red, orange or yellow. These LEDs emit light that may be all of the same color, or alternatively, of two or more different colors. These light-emitting devices are preferably arranged upon the board/lighting head substantially co-linearly. The LEDs are electrically coupled to the electrical conductors supported by the board/lighting head for selectively causing each such light-emitting device to emit light. The electrical cable coupled between the base unit and the lighting head selectively supplies electrical power from the base unit to the lighting head to selectively operate the light-emitting devices. In this manner, a user may place the lighting head behind an infant's arm, or behind another relevant portion of the infant's body, thereby directing light therethrough for enhanced viewing.

A pediatric arm board featuring an illumination function, in accordance with the present invention, may be of very simple construction. A conventional arm board contains a rigid core layer, made out of cardboard, wood or plastic. In accordance with the preferred embodiment of the present invention, a printed circuit board, upon which LEDs are mounted, is substituted for the rigid core of prior art arm boards. The printed circuit board makes the arm board sufficiently rigid, while also routing electrical conductors to the LEDs. In the preferred embodiment, the LEDs are surface-mount LEDs that are physically mounted upon an upper surface of the printed circuit board, and electrically connected to electrical conductors of the printed circuit board.

The printed circuit board may be covered, in part, by a padded cloth or foam, and enclosed in a translucent fabric sleeve surrounding the printed circuit board for resting against the infant's skin. In this case, the padding preferably has one or more openings formed therein allowing light emitted by the plurality of light-emitting devices to pass therethrough unimpeded. Alternatively, the printed circuit board may be fully encased in a flexible, transparent material, e.g., relatively-clear silicone or another thermoplastic. If desired, adhesive strips, or Velcro-brand fastening strips, may be included to wrap around the infant's arm to secure the arm board in place behind a portion of the infant's body that is to be viewed.

Preferably, the base unit includes an electrical control circuit for selectively applying electrical power to the lighting head via the electrical cable. This electrical control circuit may include user-operated controls for selecting between at least two different modes of operating the plurality of light-emitting devices to better suit the particular patient being treated. After placing an IV line in the infant's arm, the electrical cable connecting the base unit to the arm board can be disconnected from the arm board; the base unit is then ready to be connected to another arm board/light head, e.g., to place an IV line in another patient. The arm board/light head is inexpensive enough to be disposable; when the IV line is removed from the infant's body, the arm board/light head can simply be thrown away.

In an alternate embodiment of the present invention, a semi-rigid plastic housing releasably receives the lighting head in a manner which allows the lighting head to be withdrawn from such housing after venipunture is achieved. The semi-rigid housing includes a contact portion for being secured against a portion of the infant's body; at least this contact portion of the housing is translucent for allowing light emitted by the light-emitting devices to pass into the infant's body.

Another aspect of the present invention relates to a method of illuminating tissues of an infant for the purpose of a venipuncture or the like. In practicing such method, a semi-rigid elongated board is provided, and a series of light-emitting devices, for example LEDs, are supported therefrom. The elongated board is placed beneath the arm, or another limb, of an infant and secured thereto to immobilize the infant's limb. Electrical power is supplied to the light-emitting devices to illuminate the infant's limb, and the venipuncture is performed while the infant's arm is illuminated by such light-emitting devices. After the venipuncture is performed, electrical power is removed from the light-emitting devices.

In one preferred embodiment of practicing such method, electrical power is supplied to the light-emitting devices by providing a base unit that is separate from the elongated board, and which base unit includes a source of electrical power. In practicing the preferred method, an electrical cable is releasably coupled between the base unit and the elongated board for supplying electrical power from the base unit to the light-emitting devices. Removal of electrical power from the light-emitting devices preferably includes uncoupling the electrical cable from the elongated board.

In an alternate embodiment of practicing such method, a semi-rigid plastic housing is provided for releasably receiving the elongated board; this housing includes at least one contact portion for engaging the arm of the infant. This contact portion of the housing is preferably translucent, and is secured to the contact portion of the housing behind the infant's arm to immobilize the infant's arm. The elongated board is releasably secured to the housing, for example, by sliding the elongated board into the housing, for directing light into the infant's arm. After venipuncture is achieved, the elongated board is removed from the housing, and can be used in conjunction with a second housing. The original housing may be left in place secured to the infant's arm until intravenous access is no longer required, at which time the original housing may be discarded.

### BRIEF DESCRIPTION OF THE DRAWINGS

FIG. 1 is a perspective view of a preferred embodiment of a pediatric trans-illumination device constructed in accordance with an embodiment of the present invention, and having a center section partially cut-away.

FIG. 2 is a top view of the arm board portion of the trans-illumination device of FIG. 1.

FIG. 3 is a cross-sectional view of the arm board taken along line 3-3 of FIG. 2.

FIG. 4 is a cross-sectional view of the arm board taken along line 4-4 of FIG. 2.

FIG. 5 is a top view of the printed circuit board included in the arm board of FIG. 1.

FIG. 6 is a cut-away view of a second embodiment of the present invention

FIG. 7 is a top view of the pediatric trans-illumination device depicted in FIG. 6.

FIG. 8 is a cross-sectional view taken along line 8-8 of FIG. 7.

FIG. 9 is a cross-sectional view taken along line 9-9 of FIG. 7.

FIG. 10 is a top view of the printed circuit board included in the light head of FIG. 6.

FIG. 11 is a top view of a third embodiment of the present invention.

FIG. 12 is a cross-sectional view taken along line 12-12 of FIG. 11.

FIG. 13 is a top view of a padded layer included in the embodiment shown in FIG. 11.

FIG. 14 is a top view of a printed circuit board, and associated LEDs, included within the embodiment of FIG. 11.

FIG. 15 is an exploded perspective view of an alternate embodiment wherein an encased, re-usable lighting head slides into a disposable semi-rigid housing to perform a venipuncture procedure.

### DETAILED DESCRIPTION OF THE PREFERRED EMBODIMENTS

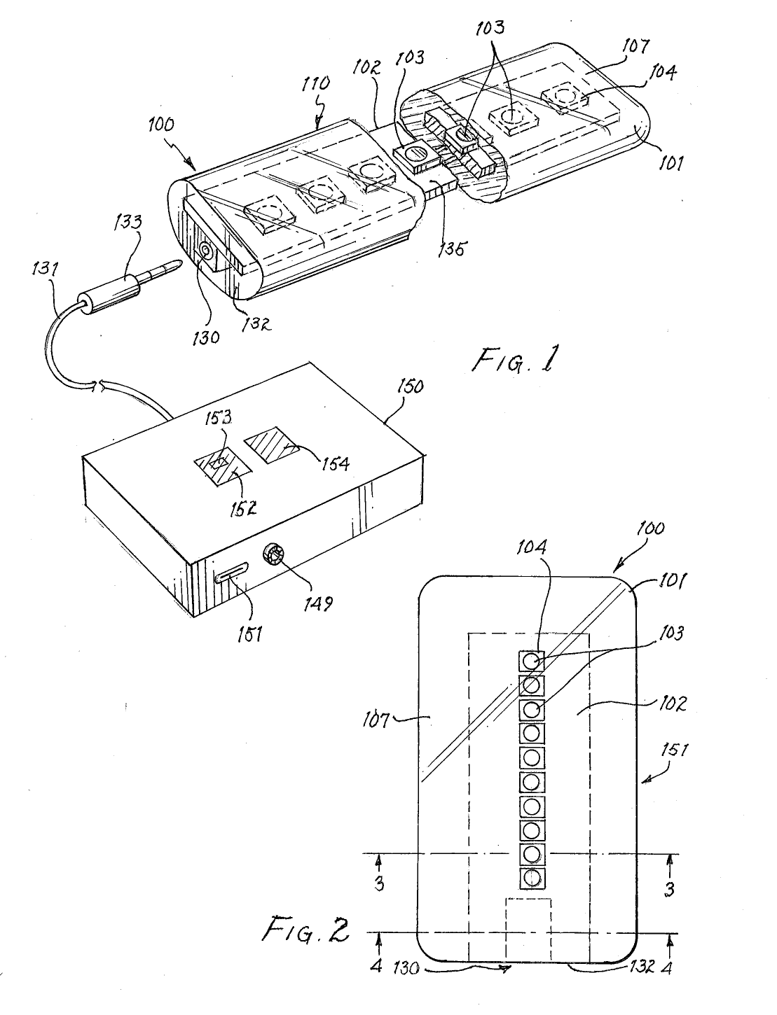

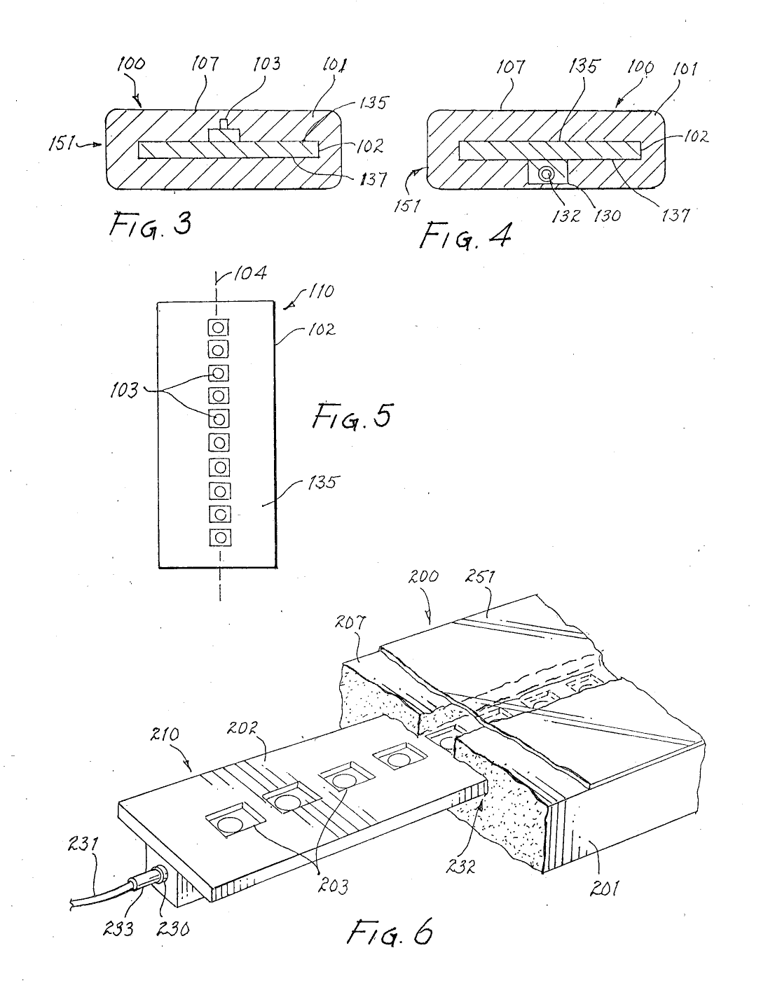

FIGS. 1-5 illustrate a first preferred embodiment of an LED-based pediatric tissue illumination device, designated generally by reference numeral 100, in accordance with a preferred embodiment of the present invention. Pediatric tissue illumination device 100 preferably includes a low cost, disposable small arm board 110. Arm board 110 includes a surrounding padded layer 101 including a top surface 107 intended for direct contact with a patient's limb. Printed circuit board (“PC board”) 102 provides physical support for, and makes electrical connections to, a series of surface mount LEDs 103, and further serves to make arm board 110 more rigid.

Padded layer 101 can be made from any material that has translucent properties or partially translucent properties, such as a soft or hard clear plastic, nylon, silicone, or another similarly transparent medium. Preferably, padded layer 101 is formed from a soft, malleable, largely-transparent, plastic material that entirely surrounds PC board 102. Depending upon its thickness and nature, plastic padded layer 101 may act in conjunction with PC board 102 to further stiffen arm board 110. A soft thermoplastic, such as Versaflex OM3060-1 offered by PolyOne GLS Thermoplastic Elastomers, is preferred when forming padded layer 101, because it can be substantially transparent, relatively soft to the touch, and has been medically tested to the standards which govern the materials used for medical devices. In the preferred disposable embodiment of the present invention illustrated in FIGS. 1-5, padded layer 101 is transparent enough to allow light emitted by LEDs 103 to pass directly therethrough, and into the patient's limb. Moreover, given the soft, malleable nature of thermoplastics like silicone, there is little risk of injury from the device having direct contact with a patient's limb. If desired, arm board 110 may be fitted within a thin, translucent fabric sleeve (not shown) to avoid irritation of the patient's skin when arm board 110 is secured against the patient's limb.

PC board 102 includes a top surface 135 and an opposing bottom surface 137 which both have electrical conductors (not shown) printed thereon in a conventional manner for connecting LEDs 103 in a series configuration. Preferably, LEDs 103 are surface-mount LEDs, and are only approximately 0.8 mm tall. LEDs 103 are secured to, and supported by, PC board 102. LEDs 103 are preferably arranged in single-file row 104. Alternatively, LEDs 103 may be clustered to concentrate the emitted light into a smaller area of PC board 102. It is preferred that LEDs 103 provide one or more colors of light in the low energy portion of the visible spectrum (i.e., red or orange) to best illuminate human flesh.

Pediatric arm board 110 may have one or more groups of LEDs 103 connected upon PC Board 102. In this case, LEDs 103 may all be of a single color, or they may be of two or more colors. A single group of parallel-connected LEDs would preferably have a common anode and a common cathode; such that they could all be driven to emit light by controlling the voltage on two conductors. If more than one parallel connected chain of LEDs is used, the chains may share their common cathode connection. Thus, for a PC Board 102 upon which two groups of parallel connected LEDs are used, the electrical connector will require three conductors, as the cathode connection is shared by all of the LEDs in both groups. If only one parallel connected group is used, the electrical connector supplying driving current to arm board 110 requires only two conductors. In the preferred embodiment, ten to twenty LEDs 103 are included on PC Board 102. Those skilled in the art can appreciate that the LEDs 103 need not be connected in a parallel fashion; they may also be connected in a series fashion or in a combination of series and parallel.

Referring again to FIG. 1, electrical driving current is supplied to arm board 110 from a base unit 150 via cable 131. Connector 133 of cable 131 is removably connected to arm board 110 into connector 130. Connectors 130 and 133 are preferably a TRS (tip-ring-sleeve) receptacle and jack respectively, as are commonly used in audio applications. Connector 130 is preferably provided on an end 132 of arm board 110 and attached to the bottom surface 137 of PC Board 102. As shown in FIG. 1, connector 130, and connector 133, preferably extend generally parallel to the parallel-connected chain of LEDs 103. Alternatively, connector 130 may be rotated 90 degrees to face either downward (relative to FIG. 1), or toward one side of arm board 110; in this event, connector 133 may then be inserted into connector 130 either upwardly from the bottom, or inwardly from one side of arm board 110, respectively, with connector 133 extending generally perpendicular to the parallel-connected group of LEDs 103.

In the case where arm board 110 includes two parallel-connected chains of LEDs 103, cable 131 includes three conductors, and terminates in a three-terminal connector 133 similar to those used for stereo-audio headphones. One terminal connects with the common cathode of arm board 110, and the other two terminals drive the two separate anodes of LEDs on arm board 110. In this instance, LEDs 103, extending linearly along row 104 of PC Board 102, preferably alternate sequentially (e.g., red, orange, red, orange, red, orange, . . . ); the red LEDs are connected in parallel with each other, and the orange LEDs are connected in parallel with each other. Of course, if arm board 110 includes only a single parallel-connected group of LEDs 103, then cable 131 need include only two conductors, and connector 133 needs only two terminals, one for coupling to the common anode, and one for coupling to the common cathode, of each of the LEDs in the group. Alternatively, if the cathodes of the two separate series-connected chains of LEDs 103 are not electrically coupled, then a four conductor cable 131 and connector 133 may be used.

Still referring to FIG. 1, battery-powered base unit 150 measures approximately 3.0″×2.1″×1.3″ in size, and may be powered by two lithium ion batteries (not shown), each of which generates a nominal voltage of 3.7V, and in combination are rated at 1.8 Amp-hours. If desired, base unit 150 may include an integrated circuit battery charger (not shown), e.g., integrated circuit type MCP73837, for charging the aforementioned lithium ion batteries.

As shown in FIG. 1, a mini-USB port 151 may be provided, if desired, on the exterior of base unit 150 for coupling to a mini-USB plug (not shown) that sources 5 volts DC to mini-USB port 151. Also shown in FIG. 1 is an optional female jack 149 adapted to receive a corresponding male jack from an AC-to-DC power adapter (not shown). Mini-USB port 151 is coupled to the input of the MCP73837 integrated circuit for sourcing 5 volts D.C. thereto. Female jack 149 may also be coupled to the input of the MCP73837 integrated circuit for sourcing from between 5-6 volts D.C. thereto. Accordingly, the lithium ion batteries of base unit 150 may be charged via mini-USB port 151 or via female jack 149. The MCP73837 IC is compatible with both the 5V mini-USB input as well as AC adapters with DC outputs of up to 6.0V. Alternatively, base unit 150 may include its own AC voltage transformer, rectifier, capacitor and voltage regulator components for converting 110 V AC power from a wall outlet to a 5-6 V DC power source.

As shown in FIG. 1, base unit 150 preferably includes a power button switch 152 and a mode select button switch 154. The power switch 152 powers the base unit on from the off state when depressed for at least 1 second. If the unit is already powered on, then depressing the power switch 152 for at least one second will power the unit off. A green LED 153 may be provided, if desired, on the front cover of the base unit to indicate that the device is powered on and/or to indicate battery charge status. Alternatively, the mode selection function could be accomplished without the addition of a dedicated button switch 154 by incorporating the mode selection functionality into the power button switch 152 by programming a microcontroller to respond differently to switch depressions of different time durations.

In the event of a low battery condition, the green LED 153 begins to blink at a 1 Hz rate in order to warn the user that the unit has approximately 45 minutes of remaining operation before it must be recharged. Recharging may be accomplished, for example, by coupling mini-USB port 151 to a mating mini-USB output plug, e.g., a USB cable plugged into a USB port of a personal computer. In the case of USB charging from a personal computer, the circuitry in the MCP73837 integrated circuit detects the output current capability of the source, which is either 100 mA or 500 mA, and sets the charging current accordingly. Alternatively, female jack 149 may be coupled to the output of an AC-to-DC adapter. A further option is to physically remove the lithium ion batteries from base unit 150 between uses and charge such batteries in a separate battery charging unit.

The mode select button switch 154 may be used to choose which of two or more predetermined user modes will be used to control LED illumination. Upon first time power-up, the base unit will default to user mode #1. Subsequent power up events will place the device in the last used state. In either case, each press of the mode select button will advance the unit to the next user mode, i.e. 1-2-3-4-1-2 . . . and so on.

For the case of arm board 110, the number of different modes can be limited. For example, in the case where arm board 110 includes one chain of red-colored LEDs, and a second chain of orange-colored LEDs, it may be sufficient to simply allow for 1) red-only operation; 2) orange-only operation; and 3) red and orange operation simultaneously. Other modes may be provided, if desired. For example, a fourth mode might illuminate the red LEDs continuously while causing the orange LEDs to gradually cycle on and off once per second in a pulsed fashion. In another optional mode of operation, two different LED colors may be held at two predetermined constant intensity levels, wherein the two intensity levels are selected to provide a desired “mix” of wavelengths particularly suited to a patient's physiology.

In order to provide such modes of illuminating LEDs 103, base unit 150 preferably includes a microcontroller (not shown). For example, an 8 bit RISC based microcontroller, such as the ATTINY167 from Atmel Corporation, may be used. The microcontroller may be programmed to enter or awake from its sleep state if the power switch 152 is depressed and held for more than one second. The microcontroller may be further programmed to advance from one pre-programmed user mode to the next if mode select button switch 154 is depressed. The microcontroller may be further programmed to provide pulse width modulated (PWM) signals which are output to LEDs 103 on printed circuit board 102 by way of cable 131, connector 133, and connector 130. One pulse width modulated signal is provided for each separate parallel-connected chain of LEDs. Within base unit 150, the PWM output signals from the microcontroller are used as enable control signals to inputs of N-channel MOSFET transistors (again, one for each separate series-connected chain of LEDs). The preferred embodiment uses SI2318DS N-channel MOSFET transistors from Vishay Corporation. The PWM signals from the microcontroller are connected to the gate pin of the MOSFET transistors for controlling the intensity and/or timing of the light emitted by the LEDs in each chain. Further details regarding the operation of the aforementioned microcontroller and MOSFET transistor drivers are provided in related co-pending patent application Ser. No. ______, entitled “MEDICAL IMAGING DEVICE”, naming the present applicants as the inventors, filed concurrently herewith, the specification and drawings of which are hereby incorporated by reference.

The plastic-encapsulated embodiment of arm board 110 shown in FIGS. 1-5 can be made relatively thin (on the order of millimeters), as both PC board 102, and surface-mount LEDs 103, have a relatively small thickness. Arm board 110 does not include its own power source, which helps to further reduce the size and cost of manufacture of arm board 110. The reduction in the size and cost of arm board 110 facilitates one-time use, making arm board 110 essentially disposable, and avoiding the need for autoclaving or other sterilization measures.

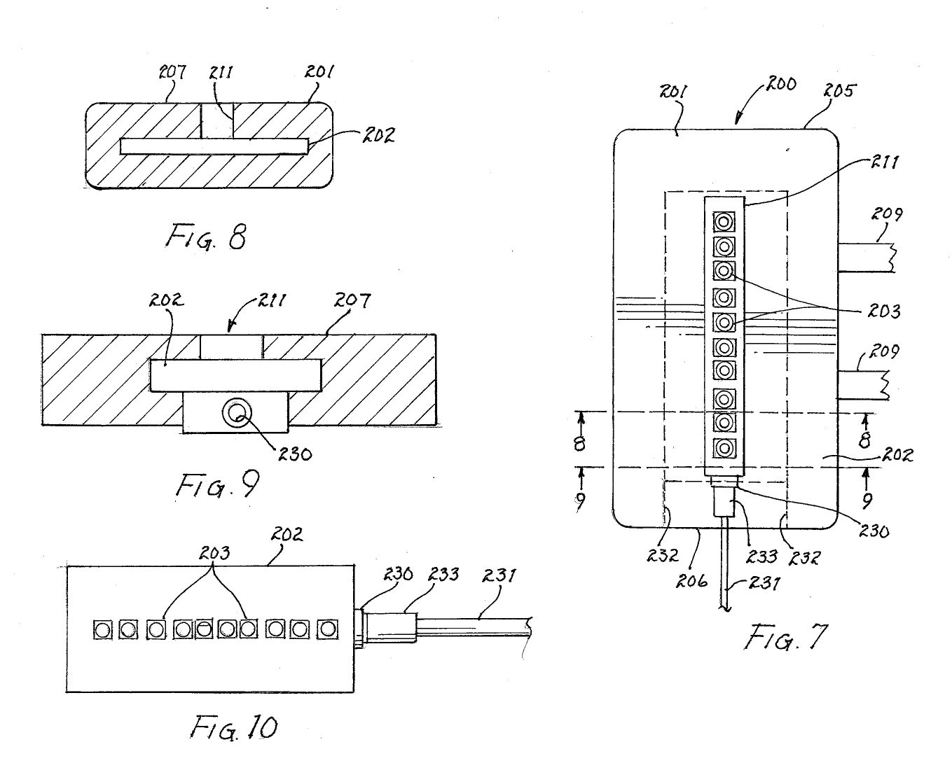

FIGS. 6-10 illustrate an alternate pediatric arm board 200. In this embodiment, LED lighting unit 210 may easily be inserted into, and removed from, foam padding 201. After use, lighting unit 210 may be removed from foam padding 201 and inserted into a fresh foam padding 201 for re-use. Alternatively, arm board 200 may be disposable and simply discarded after a single use. Within foam padding 201, a hollow channel 232 is provided for passage of lighting unit 210 into and out of foam padding 201. In FIG. 7, hollow channel 232 is indicated by correspondingly numbered dashed lines.

Lighting unit 210 includes a PC board 202 similar to PC board 102 of FIG. 1. As in the case of PC board 102, PC board 202 preferably includes a linear pattern of surface-mount type LEDs 203. Alternately, a “T-shaped” configuration or a clustered formation may be used for LEDs 203. LED light unit 210 is inserted into back end 206 of arm board 200 through channel 232, and passed toward the opposite end 205 of arm board 200. PC board 202 may itself serve as the rigid structure within pediatric arm board 200; if desired, a supplemental rigidifying member (not shown in FIGS. 6-10) may be included.

Still referring to FIGS. 6-10, LEDs 203 may be of a single wavelength (e.g., red), if desired, or two or more wavelengths (e.g., red and orange). Since padded layer 201 may be formed from a foam that is not necessarily transparent, padded layer 201 preferably includes a slot 211 formed in its uppermost face 207, and communicating with hollow channel 232 below, to allow light emitted by LEDs 203 to pass through to the patient's skin. Top surface 207 of padded layer 201 may optionally be covered by a translucent plastic panel 251, which serves to prevent any physical contact between LEDs 203 and the patient's skin. If desired, arm board 200 (including light head 210 and padded layer 201) may optionally be covered with a translucent loosely-woven fabric sleeve. Light emitted by LEDs 203 may easily pass through such fabric and into the infant's limb.

In use, arm board 200 is pressed against the underside of a limb of an infant or small child, with upper surface 207 (optionally including panel 251) in contact with the patient's skin. Slot 211 aligns with LEDs 203 to allow light to pass into the skin. Arm board 200 is positioned longitudinally along an infant's limb, allowing arm board 200 to be secured via adhesive tape or Velcro® fastening bands 209 about the limb (not shown). Light is thereby directed into and through the infant's skin when the infant is wearing arm board 200. Power is preferably supplied to LED light unit 210 by power cord 231, male connector 233, and mating female connector 230.

Following use, as noted above, lighting unit 210 may be removed from channel 232 and sterilized, if desired, for re-use. Subsequently, lighting unit 210 is simply re-inserted into fresh, sterile foam padding 201 (optionally including a fresh, sterile panel 251), and if desired, this assembly is then inserted into a fresh, sterile fabric sleeve.

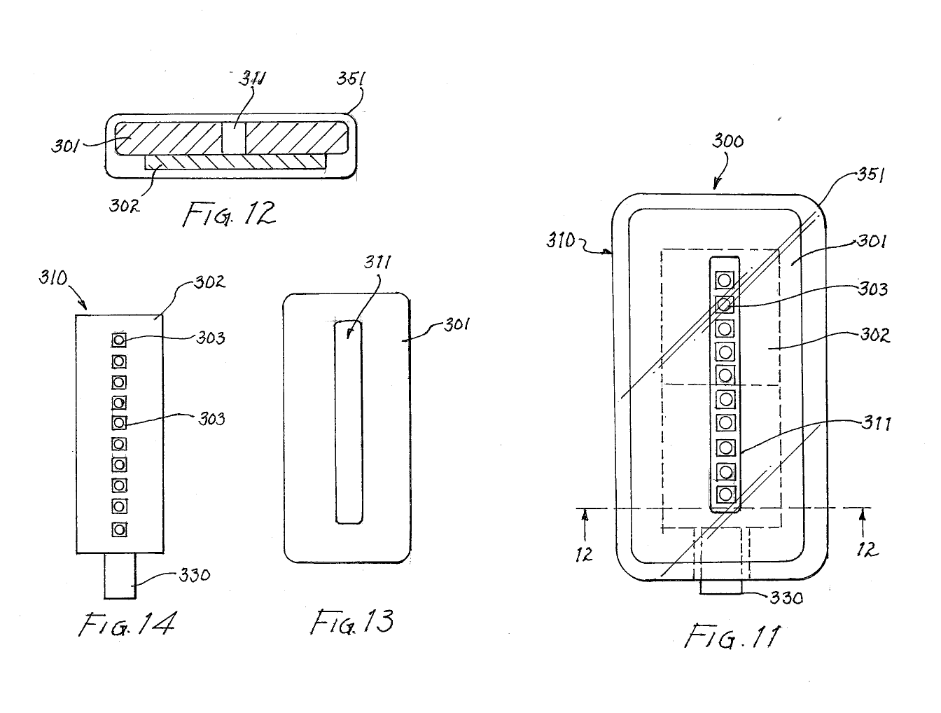

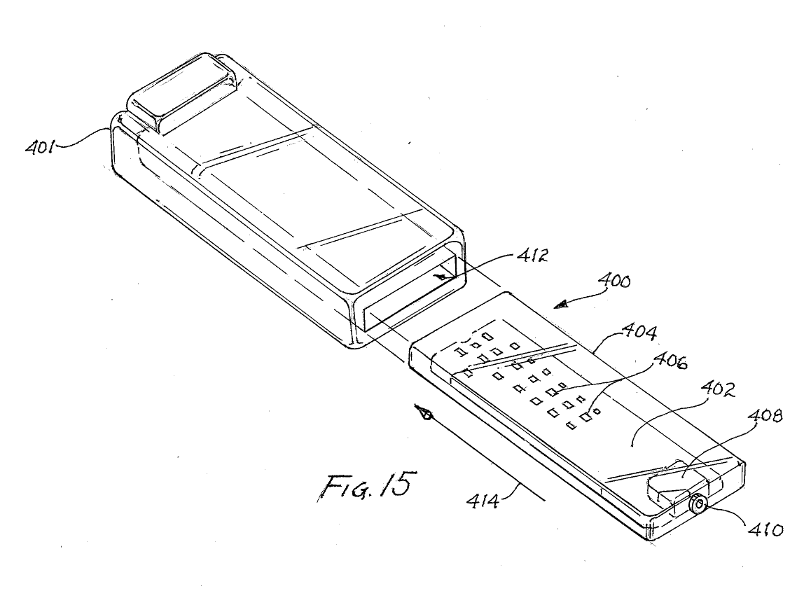

FIGS. 11-14 illustrate an alternate embodiment of an illuminated pediatric arm board 300. Arm board 300 includes a light head 310 which, like light head 110 of FIG. 1, is entirely enclosed. Light head 310 includes a PC board 302 upon which surface-mount LEDs 303 are secured and electrically interconnected. A padding layer 301 (see FIG. 13) is placed over PC board 302, and is preferably secured thereto by adhesive. Padding layer 301 includes a slot 311, or alternatively, a series of holes, for allowing light from LEDs 303 to pass therethrough.

The aforementioned structure is then inserted into a loosely-woven fabric sleeve 351. Layer 351 is preferably transparent, but is at least translucent in the region overlying LEDs 303 and slot 311 to allow the transmission of light from the LEDs 303. As in the case of the earlier embodiments, a connector 330 is provided for receiving a removable mating connector (not shown) of a power cable. Arm board 300 is preferably used once and discarded after a single use.

FIG. 15 illustrates an alternate embodiment of an illuminated pediatric arm board assembly 400. Arm board assembly 400 includes a printed circuit board 402 enclosed in a plastic housing 404 which is fully or partially transparent to light. An array of LEDs 406 is mounted to the upper surface of printed circuit board 402, while surface-mount resistors and/or other discrete components (not shown) are supported upon the underside of printed circuit board 402. In addition, electrical connector 408 is secured to the upper surface of printed circuit board 402, and includes a connector jack 410 that extends through plastic housing 404 for receiving an electrical cable (not shown).

Arm board assembly 400 can be releasably inserted into disposable plastic receptacle 401, as, for example, by sliding assembly 400 into channel 412 of receptacle 401, as indicated by arrow 414. Receptacle 401 is either partially-, or fully-transparent, to light for allowing light emitted by LEDs 406 on the upper surface of printed circuit board 402 to pass therethrough. If receptacle 401 is not fully-transparent, then at least a significant portion of receptacle 401 that lies below channel 412 is transparent, since this is the portion of receptacle 401 that will be brought in contact with the infant's body. Plastic receptacle 401 is preferably soft enough to prevent injury to the patient's skin, but rigid enough to hold the arm, or other limb, in place for IV insertion. After venipuncture is completed, arm board assembly 400 is preferably removed from channel 412 of receptacle 401; this frees-up arm board assembly for insertion within another disposable plastic receptacle for use with another infant. Meanwhile, original plastic receptacle 401 is preferably left in place to immobilize the limb during intravenous access, and is disposed of after the patient is no longer in need of IV access.

Those skilled in the art will now appreciate that a pediatric tissue illumination device has been disclosed which is useful for small children and infants. The disclosed device not only helps to illuminate veins and other tissues, but can simultaneously function as a restraint or splint. The disclosed device is portable and easy-to-use, e.g., to locate veins or other tissues on a wide variety of patients with different physiology. Moreover, the cost of the disclosed pediatric light head is so low that the illumination head of the device is effectively disposable. Alternatively, pediatric tissue illumination device can be economically produced as a re-usable light head releasably received by two or more disposable semi-rigid housings that remain with the patient following venipuncture. In addition, the disclosed pediatric tissue illumination device can be powered by a relatively simple, low cost base unit.

While the present invention has been described with respect to preferred embodiments thereof, such description is for illustrative purposes only, and is not to be construed as limiting the scope of the invention. Various modifications and changes may be made to the described embodiments by those skilled in the art without departing from the true spirit and scope of the invention as defined by the appended claims.

We claim:

1. A tissue illuminator to facilitate viewing of tissues that lie below the skin of a portion of an infant's body, comprising in combination:
a) a base unit;
b) an electrical power source housed within the base unit;
c) a lighting head, the lighting head including a semi-rigid elongated board, the board including a plurality of electrical conductors for conducting electrical signals;
d) a plurality of light-emitting devices supported by the lighting head, each light-emitting device being electrically coupled to at least one of the electrical conductors for selectively causing each such light-emitting device to emit light; and
e) an electrical cable coupled to the base unit and coupled to the lighting head for supplying electrical power from the base unit to the lighting head to selectively operate the plurality of light-emitting devices;
whereby placement of the lighting head behind a portion of the infant's body causes light to be directed into such portion of the infant's body for enhanced viewing.

2. The tissue illuminator recited by claim 1 wherein the lighting head includes a flexible cover over the board for resting against the infant's skin, the flexible cover being translucent.

3. The tissue illuminator recited by claim 2 wherein the flexible cover is relatively clear.

4. The tissue illuminator recited by claim 2 wherein the flexible cover is formed of thermoplastic.

5. The tissue illuminator recited by claim 2 wherein the flexible cover is a padded sleeve surrounding the board.

6. The tissue illuminator recited by claim 5 wherein the padded sleeve includes a layer of soft padding having at least one aperture formed therein proximate to the plurality of light-emitting devices for allowing light emitted by the plurality of light-emitting devices to pass therethrough.

7. The tissue illuminator recited by claim 1 further including at least one fastening strap for releasably securing the lighting head behind a portion of the infant's body that is to be viewed.

8. The tissue illuminator recited by claim 1 wherein the electrical cable is releasably coupled to the lighting head.

9. The tissue illuminator recited by claim 8 wherein the lighting head is disposable, and wherein the base unit is used with a plurality of lighting heads.

10. The tissue illuminator recited by claim 1 wherein the plurality of light-emitting devices are light-emitting diodes (LEDs).

11. The tissue illuminator recited by claim 10 wherein the board includes an upper surface directed toward the infant's body, and wherein the LEDs are surface mount LEDs that are physically mounted upon the upper surface of the board.

12. The tissue illuminator recited by claim 10 wherein the plurality of light-emitting devices include LEDs that emit substantially the same color of light as each other.

13. The tissue illuminator recited by claim 10 wherein the plurality of light-emitting devices includes a first LED that emits light of a first color, and a second LED that emits light of a second color substantially different from the first color.

14. The tissue illuminator recited by claim 10 wherein the plurality of light-emitting devices includes at least first and second LEDs that are electrically coupled in parallel fashion with each other along a first electrical path, and the plurality of light-emitting devices including at least third and fourth LEDs that are electrically coupled in parallel fashion with each other along a second electrical path different from the first electrical path.

15. The tissue illuminator recited by claim 14 wherein the first and second LEDs emits light of a first color, and the third and fourth LEDs emit light of a second color substantially different from the first color.

16. The tissue illuminator recited by claim 1 wherein at least one of the plurality of light-emitting devices emits light in a low energy portion of the visible light spectrum.

17. The tissue illuminator recited by claim 1 wherein the plurality of light-emitting devices are arranged upon the board substantially co-linearly.

18. The tissue illuminator recited by claim 1 wherein the base unit includes batteries for supplying electrical power to the lighting head.

19. The tissue illuminator recited by claim 18 wherein the batteries are rechargeable.

20. The tissue illuminator recited by claim 1 wherein the base unit includes an electrical control circuit for selectively applying electrical power to the lighting head through the electrical cable.

21. The tissue illuminator recited by claim 20 wherein the electrical control circuit includes at least one user-operated control for selecting between at least two different modes of operating the plurality of light-emitting devices.

22. The tissue illuminator recited by claim 1 further including a semi-rigid plastic housing for releasably receiving the lighting head, the housing including a contact portion for being secured to a portion of the infant's body, at least the contact portion of the housing being translucent for allowing light emitted by the light-emitting devices to pass into the infant's body.

23. A method of illuminating tissues of an infant for the purpose of a venipuncture, said method comprising the steps of:
a. providing a semi-rigid elongated board;
b. supporting a plurality of light-emitting devices from the elongated board;
c. placing the elongated board beneath the arm of an infant;
d. securing the elongated board to the infant's arm to immobilize the infant's arm;
e. supplying electrical power to the light-emitting devices to illuminate the infant's arm;
f. performing the venipuncture while the infant's arm is illuminated;
g. removing electrical power from the light-emitting devices after performing the venipuncture.

24. The method recited by claim 23 wherein the step of supplying electrical power to the light-emitting devices includes the steps of:
a. providing a base unit separate from the elongated board, the base unit including a source of electrical power; and
b. releasably coupling an electrical cable between the base unit and the elongated board for supplying electrical power from the base unit to the light-emitting devices.

25. The method recited by claim 24 wherein the step of removing electrical power from the light-emitting devices includes the step of uncoupling the electrical cable from the elongated board.

26. The method recited by claim 23 wherein steps c) and d) include the steps of:
a. providing a semi-rigid plastic housing for releasably receiving the elongated board, the housing including at least one contact portion for engaging the arm of the infant, the at least one contact portion of the housing being translucent;
b. securing the at least one contact portion of the housing behind the infant's arm to immobilize the infant's arm;
c. releasably securing the elongated board to the housing; and
d. removing the elongated board from the housing after performing the venipuncture.

27. The method recited by claim 26 including the steps of:
a. removing the housing from the infant's arm after intravenous access to the infant is no longer required; and
b. disposing of the housing.
